(19) United States
(12) Patent Application Publication
(10) Pub. No.: US 2008/0146987 A1
(43) Pub. Date: Jun. 19, 2008

(54) TREATMENT OF A SKIN CONDITION USING A PIPERINE-BASED COMPOSITION IN CONJUNCTION WITH IRRADIATION

(75) Inventors: Amala Soumyanath, Portland, OR (US); Antony R. Young, London (GB); Laura Faas, York (GB); Radhakrishnan Venkatasamy, London (GB); Robert C. Hider, London (GB); Susan Walker, London (GB)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: OREGON HEALTH & SCIENCE UNIVERSITY, Portland, OR (US)

(21) Appl. No.: 11/945,630

(22) Filed: Nov. 27, 2007

Related U.S. Application Data
(60) Provisional application No. 60/870,769, filed on Dec. 19, 2006.

(51) Int. Cl. A61N 1/30; A61K 31/4523; A61P 17/00; A61M 31/00

(52) U.S. Cl. 604/20; 514/321; 604/506; 604/501

(57) ABSTRACT

Embodiments of the present invention are directed to methods, apparatuses, and systems for the treatment of various skin conditions by delivery of piperine and/or a related structural analog of piperine (collectively referred to as piperine-based compounds) to an area of skin affected by a skin condition (e.g., vitiligo) in conjunction with irradiation of the affected area. Structural analogs of piperine include compounds such as THP, CHP, and RCHP. In various embodiments, irradiation of affected skin in conjunction with a piperine-based composition may result in stimulation of melanin synthesis by melanocytes which may induce pigmentation to a greater extent than with the use of the piperine-based compound alone.

### CROSS REFERENCE TO RELATED APPLICATIONS

The present application claims priority to U.S. Provisional Patent Application No. 60/870,769, filed Dec. 19, 2006, entitled “Treatment of a Skin Condition Using a Piperine-Based Composition in Conjunction with Irradiation,” the entire disclosure of which is hereby incorporated by reference in its entirety.

### TECHNICAL FIELD

Embodiments of the invention relate generally to the treatment of skin conditions, specifically to methods, apparatuses, and systems associated with the treatment of vitiligo and other skin conditions.

### BACKGROUND

Individuals afflicted with any one of various skin conditions face a multitude of hardships as a result thereof. A skin condition, unlike various other diseases and conditions, is readily apparent to others and may be difficult to hide. This conspicuousness often leads to low self-esteem and poor quality of life.

A particularly troublesome skin condition is vitiligo, affecting a staggering 1% to 2% of the world's population. This life-long condition is characterized by white patches on the skin caused by a loss of melanocytes from the affected area. Unfortunately, not only are there no universally-approved treatment regimes for vitiligo, current approaches to treating vitiligo (e.g., photochemotherapy) are unsatisfactory and often result in a mottled appearance rather than normal pigmentation. Therefore, methods, apparatuses, and systems to effectively treat skin conditions such as vitiligo are of substantial clinical importance.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1A represents a piperine molecule suitable for use in various methods in accordance with various embodiments of the present invention;

FIG. 1B represents an analog of the piperine molecule of FIG. 1A;

FIG. 1C represents an analog of the piperine molecule of FIG. 1A;

FIG. 1D represents an analog of the piperine molecule of FIG. 1A;

FIG. 1E represents an isomer of the piperine molecule of FIG. 1A;

FIG. 1F represents an isomer of the piperine molecule of FIG. 1A;

FIG. 1G represents an isomer of the piperine molecule of FIG. 1A;

FIG. 2A illustrates the dendricity of melan-a cells grown in a culture medium before exposure to piperine;

FIG. 2B illustrates the dendricity of melan-a cells grown in a culture medium after exposure to piperine in accordance with various embodiments of the present invention;

FIG. 3 outlines a series of treatment protocols, in accordance with various embodiments of the present invention;

FIG. 4 represents the pigmentation scores for mice treated with methods in accordance with various embodiments of the present invention;

FIG. 5 represents histochemical results for mice treated with methods in accordance with various embodiments of the present invention;

FIG. 6 represents the histological results for mice treated for 8 weeks with (A) vehicle alone, (B) piperine alone, and (C) piperine with UVB co-treatment, in accordance with various embodiments of the present invention;

FIGS. 7A and 7B show the different percentages of melanization of melanocytes for mice treated with UVR and compounds alone or in combination;

FIG. 8 represents the pigmentation scores for mice treated with piperine alone and with UVB radiation, in accordance with various embodiments of the present invention;

FIG. 9A represents the interaction of piperine with HSA before exposure to UVA irradiation, FIG. 9B represents the interaction of piperine with HSA after exposure to UVA irradiation, and FIG. 9C represents the interaction of piperine with HSA after exposure to SSR irradiation, in accordance with various embodiments of the present invention; and

FIG. 10A represents the interaction of piperine with DNA before exposure to UVA irradiation, FIG. 10B represents the interaction of piperine with DNA after exposure to UVA irradiation, and FIG. 10C represents the interaction of piperine with DNA after exposure to SSR irradiation, in accordance with various embodiments of the present invention.

### DETAILED DESCRIPTION OF EMBODIMENTS OF THE INVENTION

In the following detailed description, reference is made to the accompanying drawings which form a part hereof and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

A phrase in the form of “A/B” means “A or B.” A phrase in the form “A and/or B” means “(A), (B), or (A and B).” A phrase in the form “at least one of A, B and C” means “(A), (B), (C), (A and B), (A and C), (B and C) or (A, B and C).” A phrase in the form “(A) B” means “(B) or (A B),” that is, A is optional.

The description may refer to “piperine,” “analogs of piperine,” “analogs,” and “related compounds.” Such descriptions are intended to facilitate the discussion and are not intended to restrict the application of embodiments of the present invention. It is intended that the descriptions are generally interchangeable unless the context indicates otherwise.

In various embodiments of the present invention, methods, apparatuses, and systems for the treatment of various skin conditions are provided. Embodiments of the present invention are directed to delivery of piperine and/or a related structural analog of piperine (collectively referred to as piperine-based compounds) to an area of skin affected by a skin condition (e.g., vitiligo) in conjunction with irradiation of the affected area. Structural analogs of piperine include compounds such as THP, CHP, and RCHP. In various embodiments, irradiation of affected skin in conjunction with a piperine-based composition may result in stimulation of melanin synthesis by melanocytes which may induce pigmentation to a greater extent than with use of the piperine-based compound alone.

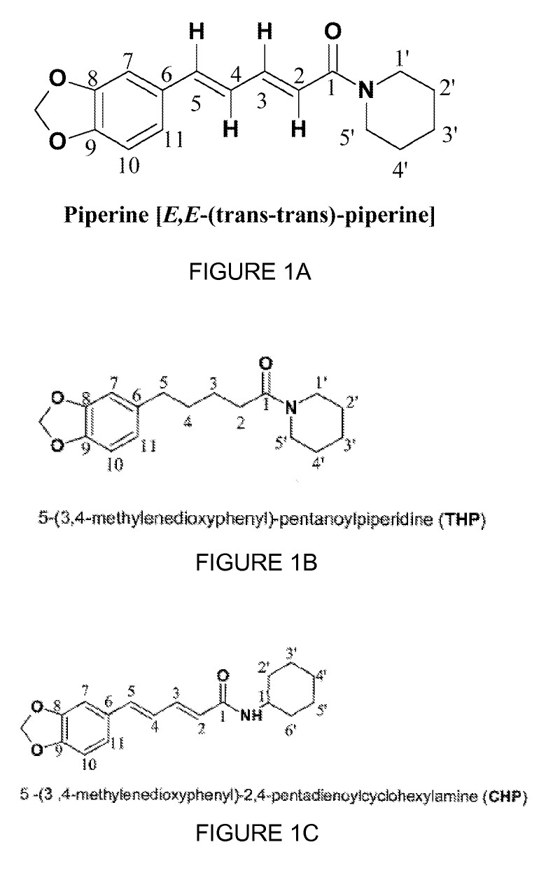

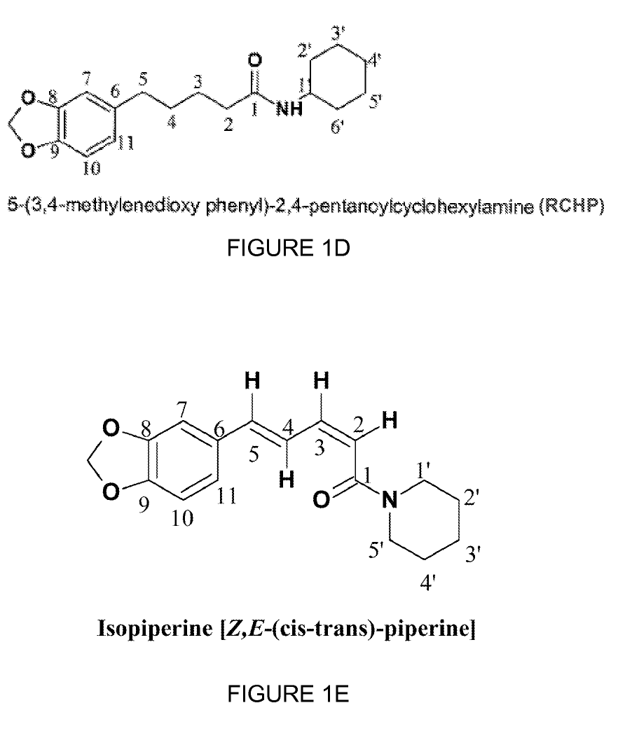

Piperine (as illustrated in FIG. 1A) is the major alkaloid of black pepper (Piper nigrum L.; Piperaceae). In various embodiments, the biologic activity of piperine, and various analogs of piperine, may be characterized by the stimulation of melanocyte proliferation and melanocytic dendrite formation. For example, as shown in FIG. 2B, piperine has been shown to stimulate melanocyte proliferation and dendrite formation in melan-a cells (an immortal pigmented mouse cell line cultured from epidermal melanoblasts from embryos of inbred C57BL mice) relative to an untreated sample as shown in FIG. 2A. This property may render piperine (and/or analogs thereof) a potential treatment for the skin depigmentation disorder, vitiligo.

Piperine alone, however, may be incapable of sufficiently stimulating melanin synthesis, melanin being generally responsible for skin pigmentation. For a skin condition such as vitiligo, which is characterized by the loss of melanocytes, stimulation of melanocyte proliferation alone may be insufficient in restoring pigmentation to the affected skin.

In an embodiment, a treatment including irradiation of an area of skin affected by a skin condition may increase effectiveness of piperine in inducing pigmentation. In various embodiments, irradiation of an area of skin treated with piperine may result in a stimulation of melanin production in melanosomes. In some embodiments, melanin may be transferred through melanocytic dendrites to epidermal keratinocytes, which may cause skin pigmentation to occur.

In various embodiments, piperine may be administered by any one or more of various routes. For example, in various embodiments, piperine may be administered by any one or more of oral, topical, intravenous, subcutaneous, or intramuscular routes. In certain preferred embodiments, piperine may be applied topically at least to the area of the skin where treatment is desired, whether currently affected by a skin condition or not.

In an embodiment, a piperine-based compound may be delivered to skin in conjunction with ultraviolet radiation (UVR). In an embodiment, the compound may be delivered to the skin within a sufficient proximity of the area of the skin having the particular skin condition (such as vitiligo) by topical, intravenous, subcutaneous, or intramuscular routes. In an embodiment, UVR may be applied to skin in the area having the particular skin condition whether the compound is applied directly to or in proximity to the affected tissue (for example, subcutaneous vs. oral).

In various embodiments, piperine may be administered in any one or more of various forms. In various embodiments, piperine may be delivered by any one or more forms including, for example, a solid powder, a paste, an ointment, a cream, a tablet, a capsule, and a solution. In an exemplary embodiment, piperine may be dissolved in dimethylsulfoxide (DMSO) and administered directly to the skin. In various ones of these embodiments, DMSO may enhance penetration of piperine into the skin. Any one or more of various other penetration enhancers suitable for the purpose may be used in addition to or instead of DMSO. In various embodiments, a penetration enhancer may increase the percutaneous absorption of topically applied piperine-based compounds by imparting an increase in skin permeability, at least temporarily. Fatty acids (such as oleic acid), glycerin, polymeric compounds (such as polyethylene glycol), diethylene glycol monoethyl ether, and polyamides are examples of suitable chemical penetration enhancers in accordance with embodiments of the present invention. In various embodiments, a penetration enhancer may be one or more physical penetration enhancers, in addition to or instead of chemical penetration enhancers, including, for example, occlusion, iontophoresis, and sonophoresis.

Frequency of piperine delivery may be determined based at least in part on any one or more of various factors. For example, frequency of delivery may be based at least in part on severity of the skin condition. In various embodiments, frequency may be based at least in part on any one or more of the age of the patient, responsiveness of the skin condition to the treatment, and the form of piperine delivery (e.g., paste, ointment, etc.).

As mentioned previously, analogs of piperine may be used for treating skin conditions. Exemplary analogs, which may be suitable for treating a skin condition, may include tetrahydropiperine (THP: 5-(3,4-methylenedioxyphenyl)-pentanoylpiperidine) (FIG. 1B), cyclohexyl piperine (CHP: 5-(3,4-methylenedioxyphenyl)-2,4-pentadienoylcyclohexylamine) (FIG. 1C), and reduced cyclohexyl piperine (RCHP: 5-(3,4-methylenedioxy phenyl)-2,4-pentanoylcyclohexylamine (FIG. 1D). Other analogs may be similarly suitable. For the purposes of describing embodiments of the present invention, the term “analog” refers to compounds that are similar in structure and/or atomic arrangement to another compound, generally maintaining similar activity levels and/or uses.

In an embodiment, compounds other than piperine-based compounds may be used in conjunction with irradiation of the skin, provided the compounds stimulate melanocyte proliferation (i.e. a melanocyte proliferation-stimulating compound).

Additional piperine-based or related compounds that may be suitable for use in embodiments include compounds described in U.S. Pat. Nos. 6,346,539; 6,680,391; and 7,122,561, and U.S. patent application Ser. No. 10/466,495, the entire contents of which are hereby incorporated by reference.

Delivery of piperine and/or related compounds to an area of skin affected by a skin condition in conjunction with irradiation of said area may be particularly efficacious in treating the skin condition. In various embodiments, this increased efficacy may be due to a stimulation of melanin synthesis by melanocytes, which may induce pigmentation of the skin.

Irradiation may be provided in any one or more of various forms. In various embodiments, irradiation may be provided by a natural or artificial light source comprising any one or more wavelengths. For example, in embodiments, any one or more of ultraviolet light (e.g., UVA, UVB, narrow band UVB), solar simulators (e.g., SSR), and natural sunlight may be enlisted.

In embodiments, irradiation may be provided in a variety of desired protocols, including one or more specific treatments and for various durations of time.

In various embodiments, UV light may be provided by one or more Bellarium SA-1-12-100 W fluorescent tubes (available from Wolff, Erlangen, Germany). In various ones of these embodiments, the tube(s) emit about 4.1% UVB (280-320 nm) and about 95.8% UVA, but the UVB accounts for 71.5% of the erythemally effective energy when biologically weighted with the human erythema spectrum. In various other embodiments, UV light may be provided by a broad spectrum UVA-I (340-400 nm) source. An exemplary broad spectrum UVA-I may be obtained from a UVASUN2000 (available from Mutzhas, Munich, Germany). In various ones of these embodiments, of the total UV output, about 98.88% thereof is UVA-I (340-400 nm), about 1.11% UVA-II (320-340 nm), and about 0.01% UVB (280-320 nm).

In various embodiments, SSR may be provided by a Solar Simulator (available from Oriel, Stratford, Conn., USA) using a 1 kW xenon arc lamp (for example, one available from ORC Lighting Products, Azusa, Calif., USA) in conjunction with a quartz collimator, quartz lens and a Schott WG320 filter (approximately 1 mm thick). In various embodiments, SSR may be provided at 12 mW/cm² scanned between 280-400 nm (UVB 280-320 nm accounting for 8.5% of total output) at an 11-cm distance from skin being treated.

Any one or more of various other light sources may be suitable for the irradiation treatment. Exemplary light sources that may be suitable for various embodiments of treating skin conditions may include UVA (320-400 nm), broad band UVB (280-320 nm), or narrow band UVB (311-312 nm), or a mixture thereof. Light may be provided by any one or more suitable lamps available for use in clinical or domestic settings including, for example, the Cooper-Hewitt PH35, PH36, and UV-84 models; the Blak-Ray UVL-13R, and UVL-9R models; and Daavlin lamps, panels, and units.

In various embodiments, irradiation may be measured one or more times during an irradiation treatment using a radiometer. In various ones of these embodiments, any one or more of an IL 442 radiometer (available from International Light, Newburytown, USA) and a wide-band thermopile radiometer (available from Medical Physics, Dryburn Hospital, Durham, UK) may be used. In various embodiments, the selected radiometer may be calibrated according to generally-known methods. For example, in various embodiments, a double-monochromator spectroradiometer (for example, one available from DM150, Bentham, Reading, UK) calibrated against a UK National Physics Laboratory (NPL) standard lamp may be used.

Systems for irradiating skin during a course of treatment for a skin condition may include various components in addition to the light source. For example, in various embodiments, an irradiation system may include any one or more of ventilation control, temperature control, and humidity control. An exemplary temperature control may include one or more fans, which may cool the skin being irradiated. Irradiation may also be administered using light panels or phototherapy cabinets, such as solariums or tanning beds/booths. These may be used, for example, in domestic, clinical, or commercial environments.

Although delivery of piperine and/or related compounds to an area of skin affected by a skin condition in conjunction with irradiation of the area may result in an increase in efficacy of piperine in inducing pigmentation, irradiation of piperine may lead to the photoisomerization of piperine. In various embodiments and as mentioned above, isomers of piperine may be less efficacious in inducing proliferation of melanocyte proliferation. Therefore, in various embodiments, it may be desirable to avoid or minimize irradiation simultaneously with application of piperine.

In various embodiments, skin affected by a skin condition may be irradiated before and/or at some time after application of piperine to the skin. In various ones of these embodiments, such “staggered” treatment may minimize isomerization of piperine resulting in an optimal treatment of the skin condition. As mentioned previously, in various embodiments, various piperine-based compounds may experience minimal photoisomerization, or no photoisomerization at all, owing at least in part to their structures (e.g., for those structures lacking a double bond). For example, THP, and RCHP may not experience photoisomerization. Thus, in various embodiments, irradiation of the skin substantially simultaneously with the application of the piperine-based compound may not impact its efficacy in treating a skin condition.

In various other embodiments, however, irradiation simultaneously with application of piperine may nonetheless induce a satisfactory level of melanocyte proliferation relative to a treatment excluding irradiation altogether. In various embodiments, such simultaneous application may in fact be desirable for various reasons. For example, simultaneous irradiation/piperine application may be convenient due to a minimization of time a patient must spend in treating a skin condition during one or more specific treatments.

### EXAMPLES

Chemicals and reagents for the following experiments were purchased from Sigma-Aldrich Co Ltd (Irvine, UK) unless stated otherwise. Piperinic acid was prepared by hydrolysis of piperine. Melan-a cells were an immortal pigmented mouse cell line, cultured from epidermal melanoblasts from embryos of inbred C57BL mice. Sub-confluent to nearly confluent melan-a cultures (passage number 26-31) were used in this study.

While the examples use mice as an exemplary animal model, the methods in accordance with embodiments of the present invention may be used with other animals, including humans. For the purposes of describing embodiments of the present invention, the term “animals” includes humans.

Male and female inbred HRA.HRII-c/+/Skh hairless pigmented mice, age-matched (8-16 weeks old), were used in the following in vivo experiments for evaluation of pigmentation induced by piperine and/or UVR.

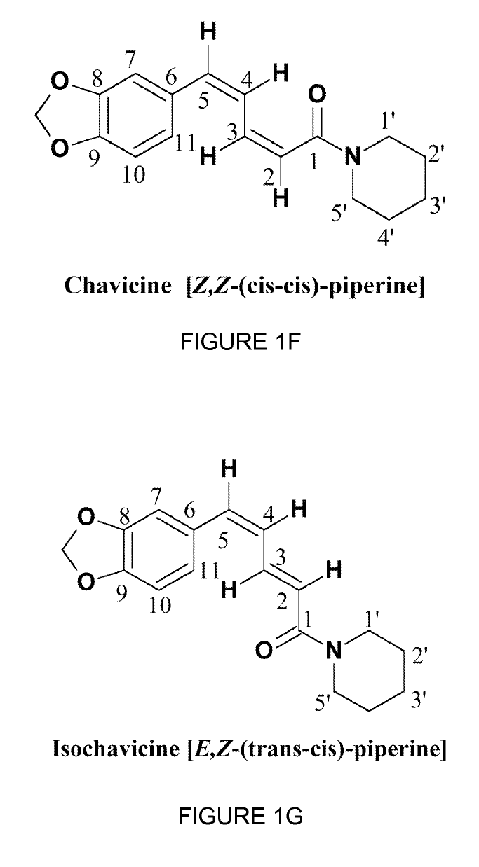

In one experiment, the compounds used included piperine, THP, CHP, and RCHP, each prepared in DMSO at a 50 mg/ml concentration, and applied to the backs of mice (n=4) twice daily on weekdays for 13 weeks, as shown in FIG. 3. As shown, Group A received treatment with compounds only from week 1-13. Group B was treated with compound from week 1-13, with additional UVB irradiation from weeks 5-13. Group F received UVB alone (no topical treatment) for 9 weeks, corresponding to weeks 5-13. Groups C, D and E were treated as Group B from weeks 1-7. All treatments was suspended for 3 weeks (8-10) and re-started as topical treatment with compounds only (Group C), UVB only (Group D), or topical treatment with UVB (Group E) from weeks 11-13.

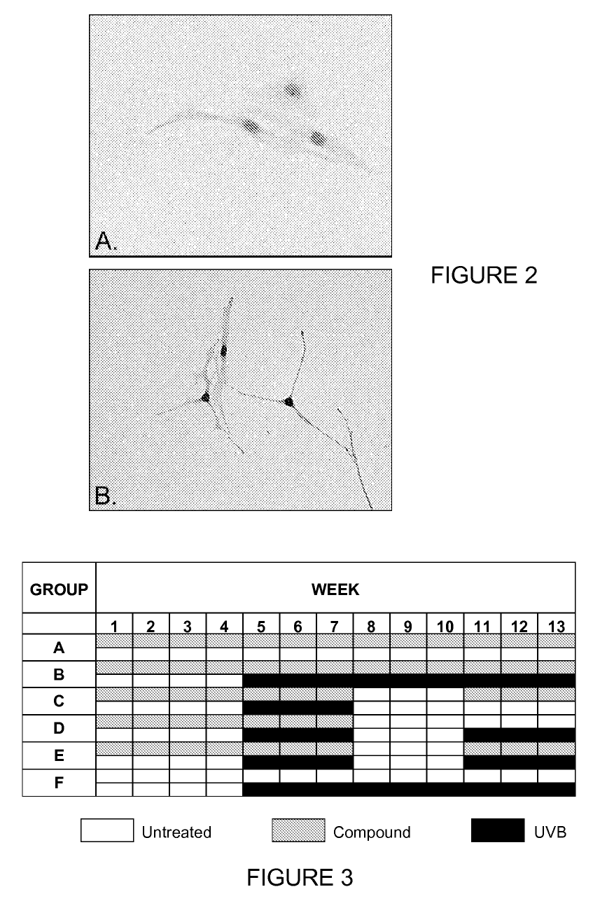

Pigmentation was assessed visually by an investigator blinded to the treatment that the animals had received using the following scoring system: 0—no pigmentation; 1—first signs of pigmentation (spots); 2—light brown; 3—medium brown; 4—dark brown; 5—black. Scores obtained at the end of each week (Friday) are shown in FIG. 4 (piperine noted as PIP). Data from equivalent treatments is combined in FIG. 4; i.e., weeks 1-4 from Groups A to E; weeks 5-7 for Groups B to E; and weeks 7-10 for Groups C to E.

As shown in FIG. 4, pigmentation increased relative to the DMSO control with use of the compounds alone, reaching a maximum score of 2 (Group A). Pigmentation was dramatically improved to score 5 with the additional use of UVB (Group B). This was greater than that achieved with UVB alone (score of about 3 for the same number of exposures) (Group F). In addition, at various points (for example, week 7), the cumulative effect of UVR and delivery of a compound appears greater than the effect of the individual treatments taken separately. Pigmentation decreased during treatment interruption in Groups C-E, weeks 8-10. However recovery was stimulated by re-treatment with compounds (Group C), UVR (Group D), or the combination of both (Group E) during weeks 11-13.

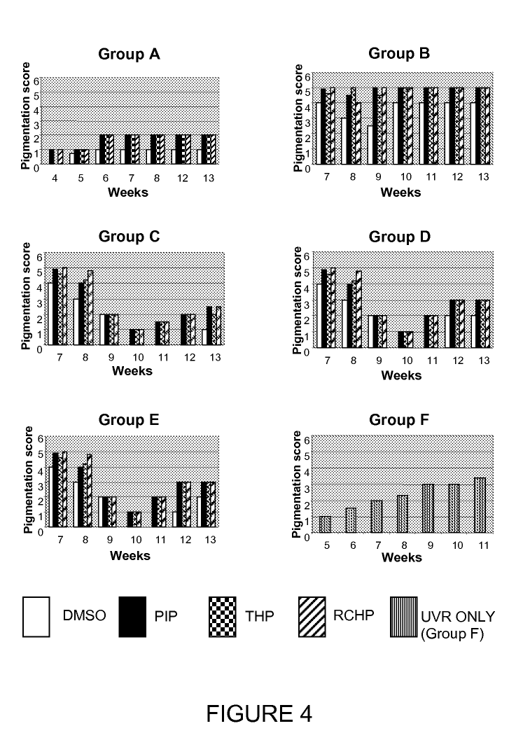

Pigmentation was also assessed histologically by DOPA staining of epidermal biopsies at the end of the 13 weeks. As shown in FIG. 5 (piperine noted as PIP), a statistically significant increase in DOPA+ cells (mean ±SD; n=4) in male (a) and female (b) mouse skin (following the protocols shown in FIG. 3) was observed, in comparison to untreated mice (low DOPA+ cell count) and controls. Treatment was continuous in control groups (Group A: Topical compound only (13 weeks), Group B: Topical (13 weeks) with UVR (weeks 5-13); Group F: UVR only (9 weeks)). For discontinuous treatment, mice were topically treated with piperine, THP, or RCHP for 4 weeks and further exposed the compound alone group to UVR from week 5-7. Treatment was then suspended for 3 weeks and re-started as topical applications only (Group C), UVR only (Group D), or topical application with UVR (Group E) during weeks 8-13. Skin biopsies were obtained at week 13. Significant differences with vehicle (P<0.05; Student's t-test) are noted by * in FIG. 5. Significant differences with groups B and D (P<0.05; Student's t-test) are noted by ** in FIG. 5. As shown, melanocyte proliferation was induced following topical application of the piperine compounds while UVB co-treatment produced more cells/mm² than application of the compound alone (Group A) or UVB alone (Group F). Greater numbers of melanocytes were seen in male than female mice for all treatment groups.

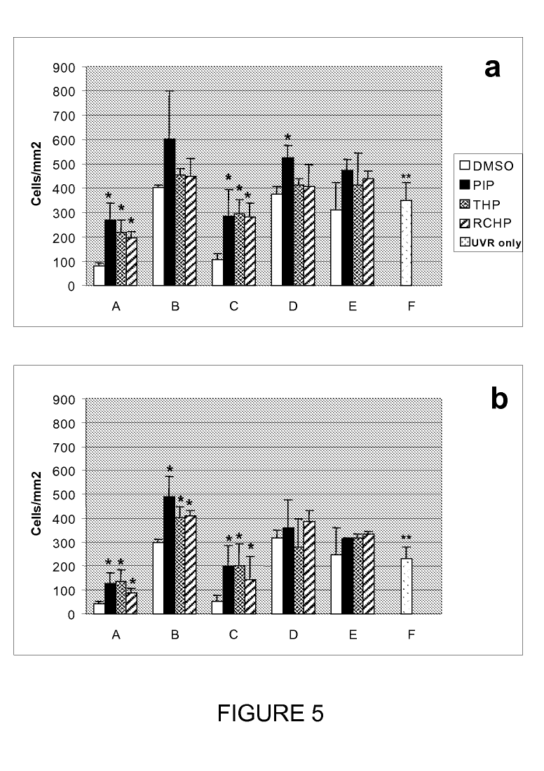

Shown in FIG. 6 is a histological comparison of skin from mice treated with vehicle alone (A), piperine alone for 8 weeks (B) or piperine with UVB co-treatment (C). As shown, melanocyte proliferation was induced following topical application of the piperine compounds while UVB co-treatment produced more cells/mm² than application of the compound alone. UVB treatment also resulted in increased melanin production in the melanocytes.

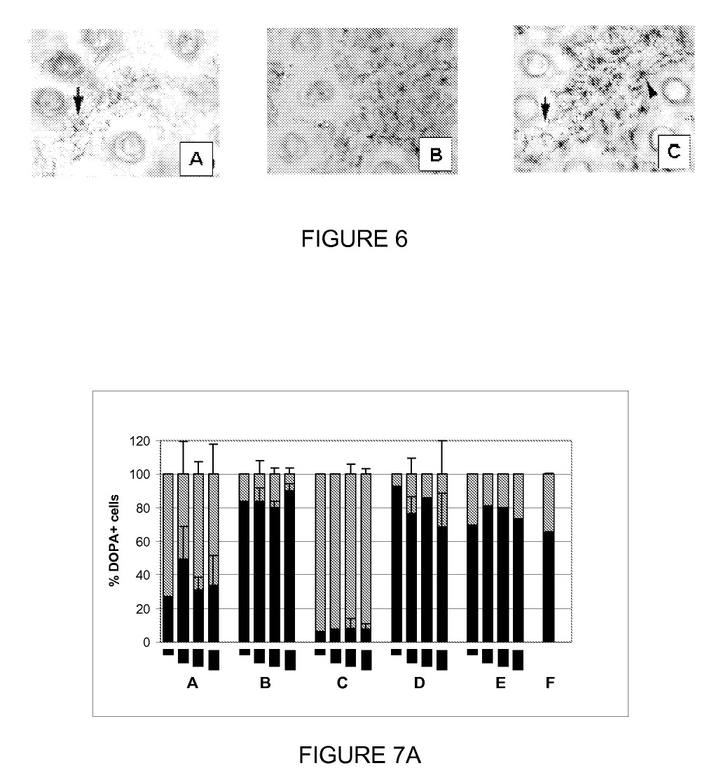

FIGS. 7A and 7B show the different percentages of melanization of melanocytes for mice treated with UVR and compounds alone or in combination. The percentage of highly (black bars) vs poorly (gray bars) melanized melanocytes in male (FIG. 7A) and female (FIG. 7B) mice are shown (treatments performed as represented in FIG. 3; n=4). Groups receiving UVR (B, D, E and F) show greater melanization than those receiving compound alone (A) or compound following treatment interruption (C) prior to histology.

In another experiment, mice (n=4 per group) were treated with (A) DMSO for 9 weeks, (B) piperine (175 mM) dissolved in DMSO for 9 weeks, (C) piperine in DMSO for 9 weeks with UVR from weeks 5-9, or (D) UVR only for 5 weeks. Piperine solution and DMSO were applied with a micropipette (100 μl) on dorsal skin twice a day (weekdays) with an interval of 5 to 6 hours between applications. UVR was administered as described below. For group (C), the irradiations were carried out every Monday, Wednesday and Friday immediately prior to the first daily application of piperine to avoid photodegradation of piperine.

The UVR source was a bank of eight Bellarium SA-1-12-100 W fluorescent tubes, and irradiations were carried out in a purpose-built unit with ventilation, temperature and humidity controls. The irradiance was monitored daily immediately before irradiations with an International Light radiometer equipped with UVR sensors. Irradiance at mouse level was typically about 0.16 mW/cm². Animals were irradiated unrestrained in metal cages with a dose of 354 mJ/cm² that was further confirmed to be sub-inflammatory from a single exposure (increase in SFT<10%; data not shown). Irradiations lasted for a maximum of 1 hour, and the positions of cages were systematically rotated to ensure even UVR exposure.

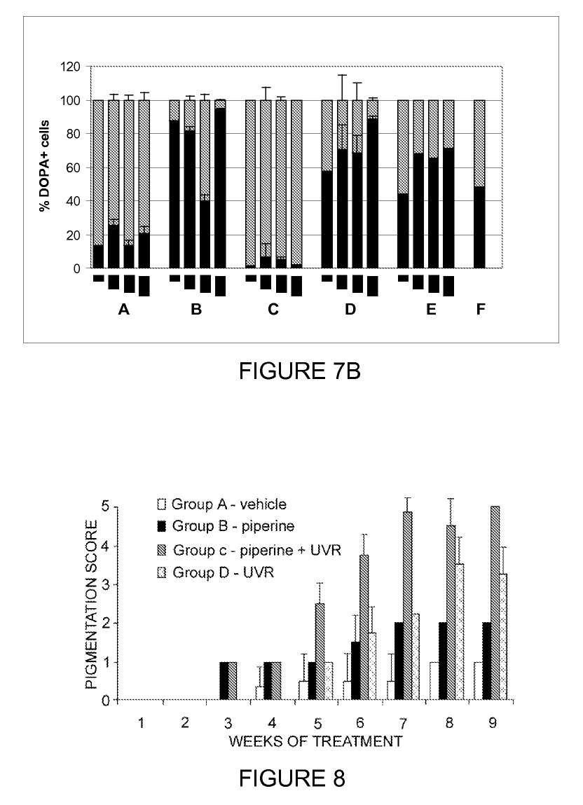

Pigmentation was assessed visually by an investigator blinded to the treatment that the animals had received using the following scoring system: 0—no pigmentation; 1—first signs of pigmentation (spots); 2—light brown; 3—medium brown; 4—dark brown; 5—black. Scores obtained at the end of each week (Friday) are shown in FIG. 8. Differences between treatment groups across the entire treatment period were compared by the Mann-Whitney U-test and found to be significant (P<0.05).

For cell culture experiments, melan-a cells were inoculated (100 μl; 6×10³ cells per well) with a repeater-pipetter into 96 well microtitre plates (nunc, Cambridge) and incubated at 37° C. in a 10% CO₂, 90% air humidified atmosphere for 4 hours. Piperine was dissolved in methanol and the solutions were sterilized by filtration (pore size 0.2 μm) and then diluted with the cell culture medium to give a final stock solution of 30 μM piperine and a non toxic concentration of methanol. Each plate was subdivided into sections each consisting of two adjacent columns of 6 wells each for piperine (10 μM; 50 μl of stock solution) or control (50 μl medium only), with a gap of 2 empty columns between each section.

The plates, placed on ice, were positioned under the irradiation sources UVA and SSR. In order to maintain sterility of the cell cultures, irradiations were carried out with the microplate lid in place. The lid reduced the UVB and UVA irradiances of the SSR source by about 45% and 30% respectively, and the UVA irradiance of the UVA source by about 20%. Microplates were irradiated with UVA doses ranging from 0-124 J·cm⁻² using the Mutzhas UVASUN2000 broadband UVA source. This represents a dose of less than 2 minimal erythema doses (MED) for sun sensitive skin type II after correction for absorption by the 96-well plate lid. Different doses were achieved by exposing particular sections of the plate (each consisting of one row of piperine exposed cells and one row of control cells) for different time periods (0, 7, 13, 22 or 28 minutes; n=6). Unexposed areas were covered with a piece of cardboard. Another set of microplates was irradiated with SSR doses ranging from 0-15 J·cm⁻² using a solar simulator for 0, 5, 7, 15 or 21 minutes (n=6). This represents doses of about 1.5 MEDs for skin type II after corrections for the effect of the microplate lid. All plates were incubated at 37° C. in a 10% CO₂, 90% air humidified atmosphere incubator for 4 days.

Cell proliferation was measured as follows. After 4 days, incubation cells were fixed using cold trichloroacetic acid solution, incubating at 4° C. for 1 hour. After washing with tap water to remove acid, medium and dead cells, plates were dried in air, and SRB dye was added. At the end of the staining period (30 minutes) unbound SRB was removed by washing with acetic acid and air drying. Cell-bound dye was solubilized in Tris [tris(hydroxymethyl)aminomethane] base and absorbance was read at 550 nm in a microplate spectrophotometer (Spectromax 190 Molecular Devises, Softmax pro version 2.2.1, 1998).

Potential binding of piperine to protein and DNA before and after irradiation was investigated using circular dichroism (CD). Deoxyribonucleic acid (DNA) and human serum albumin (HSA) binding of piperine was monitored using a 5:1 DNA base-pair: drug or protein: drug molar ratio, using piperine (100 μM) and DNA or HSA (500 μM), all made up in 1% methanolic phosphate buffer (pH 7.4). Mixtures were made of piperine with buffer, DNA solution or HSA solution. Pure solutions of piperine, DNA and HSA and mixtures were divided into three sets with one set left unexposed, the second set exposed to UVA for 5 minutes (22 J·cm⁻²) and the third set exposed to SSR for 21 minutes (15 J·cm⁻²). These solutions were then stored in a refrigerator for 3 days prior to CD (Jasco J-600 spectropolarimeter) and LD (Jasco J-720 spectropolarimeter) analysis. The irradiated samples were analyzed by UV spectroscopy (Perkin-Elmer Lambda-2 UV/VIS spectrometer) immediately after irradiation and after three days storage in a refrigerator. No changes were found to have occurred during the storage period (results not shown). CD spectroscopy was performed using a CD cell of 1 cm pathlength. LD analysis was performed to validate CD experiments on piperine binding to DNA.

Cell growth of a sample treated with 10 μM piperine relative to an untreated control sample was shown to be detrimentally affected by exposure to ultraviolet-A (UVA), which may be due to some extent to isomerization of piperine. Similar, though less drastic, results were seen upon exposure to simulated solar radiation (SSR). The difference between the results may be explained by lower levels of isomerization using SSR as compared to UVA.

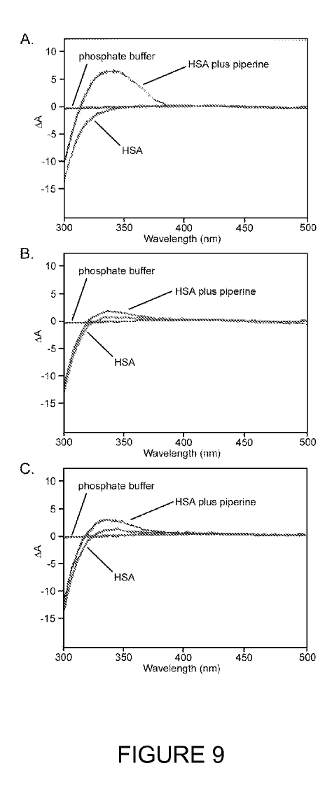

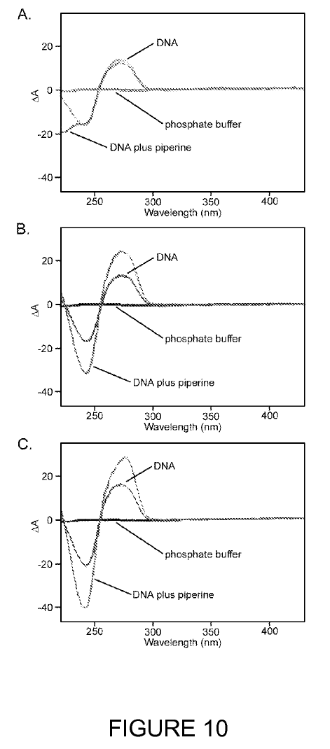

Binding of piperine to protein may also be detrimentally affected by structural changes to piperine induced by irradiation. Piperine has been found to be optically inactive, but if bound to a chiral substance, such as human serum albumin (HSA) or deoxyribonucleic acid (DNA), optical activity of piperine may be induced and a signal will be observed in the CD spectrum associated with piperine's UV absorption λₘₐₓ (about 340 nm). Piperine was found to bind to HSA (FIG. 9A) but on exposure to UVA (FIG. 9B) or SSR (FIG. 9C), this was abolished, suggesting that the structural changes induced by radiation were detrimental to protein binding of the molecule. No binding to DNA was observed by CD either before or after irradiation (FIGS. 10A-10C); the increase in absorbance at 275 nm (FIG. 10B, FIG. 10C) is due to a concentration effect (solvent evaporation). Absence of binding to DNA was confirmed using linear dichroism (data not shown). This suggests that, unlike the situation with psoralen skin treatment, piperine and its isomers do not bind DNA and would not form photoadducts in vivo.

Thus, topical treatment of HRA/Skh-II mice with PIP or two of its analogs, THP and RCHP, stimulate the development of even skin pigmentation in vivo after continuous topical application, for example for four or more weeks. The darkening of skin in treated areas corresponds with an increase in the number of DOPA+ melanocytes. Animals treated with PIP or analogs prior to UVR exposure showed more rapid and darker pigmentation than those treated with UVR exposure or compounds alone. The pigmentation observed with the compounds in conjunction with UVR was also even, in contrast to the speckled perifollicular pattern observed with UVR alone in these mice, or clinically with the use of radiation based treatments. Topical treatment in combination with low dose UVR thus significantly enhances the pigmentation response with results that are cosmetically better compared to conventional vitiligo therapies. Although fading may occur when treatment is interrupted, a good pigmentation response is readily re-achieved after short periods of re-treatment. These results highlight the benefits of these compounds for use in treatments for vitiligo. Further, supplementing UVR with these compounds may offer a means of reducing overall UVR exposure in vitiligo therapy, thereby reducing the risks of developing skin cancer.

The degree of skin pigmentation is a consequence of both number of melanocytes and their degree of melanization. UVR, for example, stimulates both melanocyte proliferation and melanin synthesis. The relatively low pigmentation scores in the absence of UVR and low degree of melanization of DOPA+ cells observed in skin treated with compounds alone suggests that these compounds stimulate melanocyte proliferation rather than melanin synthesis. Re-treatment with compounds alone induced a higher difference in DOPA-positive cell number between compound and vehicle than did re-treatment with UVR alone. This result suggests that the primary effect of piperine is to stimulate rapid melanocyte proliferation and population of epidermal areas.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A method for treating a skin condition in an animal, comprising:
providing at least one piperine-based composition to skin having the skin condition; and
irradiating the skin.

2. The method of claim 1, wherein said providing at least one piperine-based composition comprises providing piperine to the skin.

3. The method of claim 1, wherein said providing at least one piperine-based composition comprises providing one or more analogs of piperine to the skin.

4. The method of claim 3, wherein said providing one or more analogs of piperine to the skin comprises providing tetrahydropiperine to the skin.

5. The method of claim 3, wherein said providing one or more analogs of piperine comprises providing reduced cyclohexyl piperine to the skin.

6. The method of claim 1, wherein said providing at least one piperine-based composition comprises providing at least one of piperine, tetrahydropiperine, cyclohexyl piperine, or reduced cyclohexyl piperine to the skin.

7. The method of claim 1, wherein said providing at least one piperine-based composition comprises providing a piperine-based composition including substantially no isomers of piperine.

8. The method of claim 7, wherein said providing at least one piperine-based composition including substantially no isomers of piperine comprises providing a piperine-based composition including substantially no isopiperine, chavicine, or isochavicine.

9. The method of claim 1, wherein said providing at least one piperine-based composition comprises providing an isomer of piperine to skin.

10. The method of claim 1, wherein said providing at least one piperine-based composition comprises providing a solid powder, a paste, an ointment, a cream, a tablet, a capsule, or a solution to the skin.

11. The method of claim 1, wherein said providing at least one piperine-based composition comprises providing the piperine-based composition to skin by injection or topical application of the composition.

12. The method of claim 1, wherein said providing at least one piperine-based composition further comprises providing a penetration enhancer to the skin.

13. The method of claim 12, wherein said providing the penetration enhancer comprises providing DMSO, a fatty acid, glycerin, a polymeric compound, diethylene glycol ethylene monoethyl ether, or a polyamide to the skin.

14. The method of claim 12, wherein said providing the penetration enhancer comprises providing an occlusion technique, an iontophoresis technique, or a sonophoresis technique to the skin.

15. The method of claim 1, wherein said providing at least one piperine-based composition to skin having the skin condition comprises providing at least one piperine-based composition to skin having vitiligo.

16. The method of claim 1, wherein said irradiating the skin comprises irradiating the skin to which the piperine is provided.

17. The method of claim 1, wherein said irradiating the skin comprises irradiating the skin prior to said providing at least one piperine-based composition to the skin.

18. The method of claim 1, wherein said irradiating the skin comprises irradiating the skin simultaneously with said providing at least one piperine-based composition to the skin.

19. The method of claim 1, wherein said irradiating the skin comprises irradiating the skin after said providing at least one piperine-based composition to the skin.

20. The method of claim 1, wherein said irradiating the skin comprises irradiating the skin with ultraviolet radiation from an ultraviolet light source.

21. The method of claim 1, wherein said irradiating the skin comprises irradiating the skin with simulated solar radiation.

22. A method for treating a skin condition in an animal, comprising:
providing at least one melanocyte proliferation-stimulating composition to skin having the skin condition; and
irradiating the skin.
